(19) United States
(12) Patent Application Publication
(10) Pub. No.: US 2019/0091608 A1
(43) Pub. Date: Mar. 28, 2019

(54) FLUID DEVICE

(71) Applicant: NIKON CORPORATION, Tokyo (JP)

(72) Inventors: Naoya ISHIZAWA, Saitama-shi (JP); Naoko TOMINAGA, Chiba-shi (JP); Taro UENO, Tokyo (JP)

(21) Appl. No.: 16/205,062

(22) Filed: Nov. 29, 2018

Related U.S. Application Data
(63) Continuation of application No. PCT/JP2017/020957, filed on Jun. 6, 2017.

(30) Foreign Application Priority Data
Jun. 6, 2016 (JP) 2016-112690

(51) Int. Cl. B01D 19/00; B01D 71/36; G01N 1/40

(52) U.S. Cl. CPC B01D 19/0031; G01N 33/49; G01N 1/405; B01D 71/36

(57) ABSTRACT

A fluid device includes a flow path through which a liquid flows, and a tank connected to the flow path and in which the liquid is accommodated. The tank includes an exhaust port through which a gas is discharged, a gas-liquid separation filter that suppresses outflow of the liquid from the exhaust port, and an absorber disposed in the tank and that absorbs the liquid.

### CROSS-REFERENCE TO RELATED APPLICATION

Priority is claimed on Japanese Patent Application No. 2016-112690, filed Jun. 6, 2016. The present application is a continuation application of International Application PCT/JP2017/020957, filed on Jun. 6, 2017. The contents of the above applications are incorporated herein.

### BACKGROUND

### Technical Field

The present invention relates to a fluid device.

In recent years, the development of a fluid device referred to as micro-total analysis systems (μ-TAS) with an aim of high-speed, high efficiency, and integrated testing in the field of extracorporeal diagnosis or micro-miniaturization of analysis equipment has attracted attention, and active research thereon is underway worldwide (for example, see Japanese Unexamined Patent Application, First Publication No. 2007-3268).

μ-TAS is superior in comparison with analysis equipment in the related art in that measurement and analysis can be performed with a small amount of specimen, and that systems are portable, and disposable due to having a low cost, and so on. Further, μ-TAS is drawing attention as a method with high usefulness when expensive reagents are used or when small amounts of multiple-specimen are analyzed.

### SUMMARY

An embodiment provides a fluid device including a flow path through which a liquid flows, the fluid device including: a tank connected to the flow path and in which the liquid is accommodated, the tank including an exhaust port from which gases are discharged; a gas-liquid separation filter that suppresses outflow of the liquid from the exhaust port; and an absorber that is disposed in the tank and that absorbs the liquid.

An embodiment provides a fluid device including a flow path, the fluid device including: a tank that includes an exhaust port from which gases are exhausted and an introduction port connected to the flow path, and in which a liquid flowing from the introduction port is accommodated; a gas-liquid separation filter that is disposed at a position of the exhaust port or in a vicinity of the exhaust port and that suppresses outflow of the liquid; and an absorber that absorbs the liquid, wherein the tank is divided into a first side into which the liquid flows and a second side from which the gas is exhausted, with reference to the gas-liquid separation filter, and the absorber is disposed at the first side of the tank.

### BRIEF DESCRIPTION OF THE DRAWINGS

FIG. 1A is a cross-sectional view showing an example of a tank included in a fluid device of an embodiment.

FIG. 1B is a cross-sectional view showing an example of a tank portion of the fluid device according to the embodiment.

FIG. 1C is a cross-sectional view showing an example of a tank portion of the fluid device according to the embodiment.

FIG. 1D is a cross-sectional view showing an example of a tank portion of the fluid device according to the embodiment.

FIG. 2 is a plan view schematically showing an example of the fluid device of the embodiment.

FIG. 3 is a photograph showing a tank portion of a fluid device of an experiment example.

### DESCRIPTION OF THE EMBODIMENTS

Hereinafter, the present embodiment will be described in detail with reference to accompanying drawings as needed. In the drawings, the same or corresponding parts are denoted by the same or corresponding reference numerals, and redundant description is omitted. Further, dimensional proportions in the respective figures may be exaggerated for the sake of explanation, and may not necessarily be the same as actual dimensional proportions.

A fluid device of an embodiment is a fluid device including a flow path through which a liquid flows, and includes a tank connected to the flow path and in which the liquid is accommodated. The tank includes an exhaust port through which a gas is discharged, a gas-liquid separation filter disposed to cover the exhaust port and configured to suppress outflow of the liquid from the exhaust port, and an absorber disposed inside the tank and configured to absorb the liquid.

According to the fluid device of the embodiment, a liquid such as a waste liquid or the like can be accommodated substantially to the full maximum capacity of a tank. In addition, in the fluid device of the related art, while it may be difficult to accommodate the liquid to the capacity of the tank fully according to a direction in which the fluid device is used, according to the fluid device of the embodiment, the liquid such as a waste liquid or the like can be accommodated to the capacity of the tank fully regardless of the direction in which the fluid device is used. Furthermore, when a biological sample such as human blood is used in a fluid device, while from the viewpoint of preventing infectious diseases or the like, it is preferable that liquid (for example, a waste liquid containing a biological sample such as blood) do not leak out of the fluid device, leakage of liquid from the fluid device of this embodiment to the outside of the fluid device can be prevented by providing a gas-liquid separation filter.

In the embodiment, “the tank (the tank section)” may be referred to as “a waste liquid tank,” “a waste liquid collecting section,” “a liquid accommodating section,” or the like.

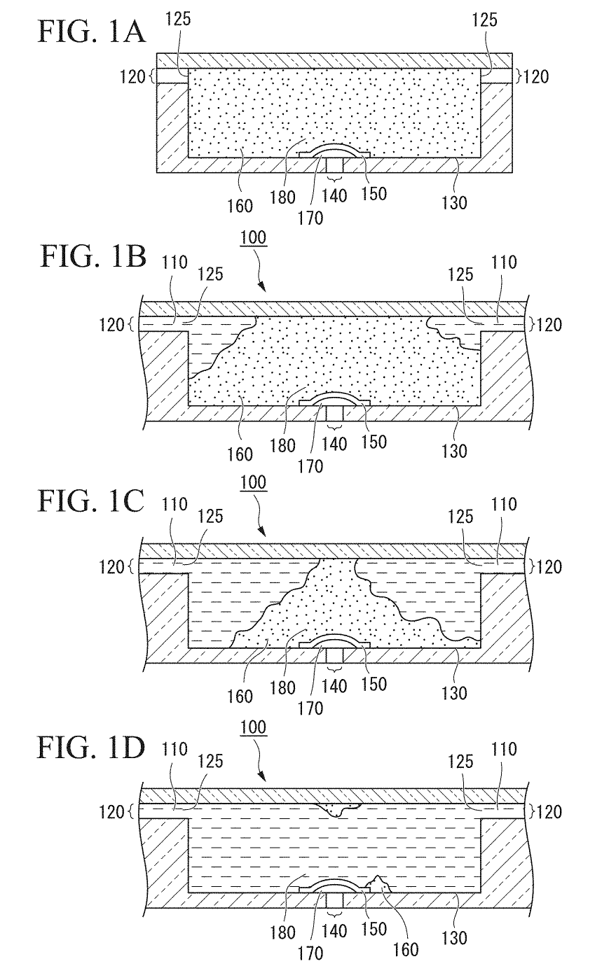

FIG. 1A is a cross-sectional view showing an example of a tank included in the fluid device of the embodiment. As shown in FIG. 1A, a tank 130 includes an exhaust port 140 through which a gas such as air or the like present in the tank 130 is discharged, a gas-liquid separation filter 150 disposed to cover the exhaust port 140 and configured to suppress outflow of a liquid 110 from the exhaust port 140, and an absorber 160 disposed in the tank 130 and configured to absorb the liquid 110. The tank 130 is incorporated and used in the fluid device. For example, the tank 130 is connected to the fluid device by a flow path 120.

As shown in FIG. 1A, the inside of the tank 130 (a space in the tank 130) may be divided into a first space 170 and a second space 180 by the gas-liquid separation filter 150. In this case, the exhaust port 140 is installed in the first space 170, and the absorber 160 is installed in the second space 180. In addition, here, the gas-liquid separation filter 150 may be adhered to the exhaust port 140. In this case, a volume of the first space 170 is very small.

In the tank 130, the position at which the gas-liquid separation filter 150 is disposed may be closer to the exhaust port 140 than a position at which the absorber 160 is disposed.

For example, the absorber 160 may be disposed such that at least a portion thereof abuts an inner surface of the tank 130. In addition, the absorber 160 may be disposed such that at least a portion thereof abuts the gas-liquid separation filter 150. The absorber 160 may be disposed in the tank 130 such that, for example, the absorber 160 covers at least an opening section of the flow path 120 so that the liquid flowed into the tank 130 from the flow path 120 does not directly come in contact with the gas-liquid separation filter 150. In addition, the absorber 160 may be disposed in the tank 130 to cover at least the gas-liquid separation filter 150. In addition, there may a region in which the absorber 160 is not present in the tank 130. In addition, the absorber 160 disposed in the tank 130 may be a single whole, or may have cuts formed in a main body thereof (for example, gaps formed in a portion of the main body), and may be divided into a plurality of parts.

FIGS. 1B to 1D are cross-sectional views showing an example of the fluid device of the embodiment including the above-mentioned tank 130. As shown in FIGS. 1B to 1D, a fluid device 100 includes the flow path 120 through which the liquid 110 flows, and the tank 130 connected to the flow path 120 and in which the liquid 110 is accommodated. A connecting portion between the flow path 120 and the tank 130 may be referred to as an introduction port 125 of the liquid 110.

The liquid 110 may be introduced into the tank 130 by a valve, a pump, or the like, included in the fluid device 100. In addition, the fluid device 100 includes a suction port connected to a suction section (a suction mechanism of the outside) configured to suction a gas in the tank 130 from outside of the tank 130, and the exhaust port 140 may be connected to the suction port via a flow path. For example, when the liquid 110 is suctioned from the suction section, the liquid 110 may be introduced into the tank 130. For example, a suction pump or the like may be exemplified as the suction section. When the suction section is connected to the exhaust port 140, the exhaust port 140f may also be referred to as a suction port 140. For example, the exhaust port 140 and the suction port 140 may be configured to be integrated.

In FIGS. 1B to 1D, in a state in which the fluid device 100 is used in a direction in which the exhaust port 140 formed at a lower side of the tank 130 is disposed downward in the tank 130 (in the direction of gravity), the liquid 110 is introduced into the tank 130. In this state, the liquid 110 introduced into the tank 130 is absorbed by the absorber 160.

FIG. 1B shows a state immediately after introduction of the liquid 110 into the tank 130 is started. In FIG. 1B, the liquid 110 has flowed into the tank 130 through the introduction port 125, and the absorber 160 in the vicinity of the introduction port 125 has absorbed the liquid 110. In addition, the absorber 160 in the vicinity of the exhaust port 140 has not absorbed the liquid 110 yet. A gas (for example, air) in the tank 130 displaced by inflow of the liquid 110 passes through the gas-liquid separation filter 150, and is discharged outside of the tank 130 through the exhaust port 140.

Here, as described above, the liquid 110 may be introduced into the tank 130 by connecting the suction section to the exhaust port 140 and suctioning the gas in the tank 130 using the suction section. Also in this case, the gas in the tank 130 passes through the gas-liquid separation filter 150 and is discharged outside of the tank 130 through the exhaust port 140.

FIG. 1C shows a state in which a larger amount of liquid 110 than in FIG. 1B is accommodated in the tank 130. In FIG. 1C, while a region of the absorber 160 that has absorbed the liquid 110 is increased and is larger than in FIG. 1B, a region that has not absorbed the liquid 110 yet is still present.

FIG. 1D shows a state in which a larger amount of liquid 110 than in FIG. 1C is accommodated in the tank 130. In FIG. 1D, a region of the absorber 160 that has absorbed the liquid 110 is increased and is larger than in FIG. 1C, and there is almost no region that has not absorbed the liquid 110 yet remaining. For example, the liquid 110 is accommodated to the capacity of the tank 130 fully at a high filling rate (a high filling rate state of the liquid 110 in the tank 130).

In a region contacting with the liquid 110 among the gas-liquid separation filter 150 shown in FIG. 1D, the gas in the tank 130 cannot be easily discharged. For this reason, as shown in FIG. 1D, in the tank 130 in a state in which almost all or all of the gas-liquid separation filter 150 is in contact with the liquid 110, it is difficult to accommodate more liquid 110.

In the fluid device of the embodiment, since the gas-liquid separation filter 150 or the like is provided, a leakage of the liquid 110 out of the fluid device can be suppressed. In FIGS. 1A to 1D, while the gas-liquid separation filter 150 is disposed in the tank 130, the gas-liquid separation filter 150 may be disposed on an outer side of the tank 130 to cover the exhaust port 140.

In addition, a position of the exhaust port 140 is not particularly limited as long as the gas such as air present in the tank 130 can be discharged. For example, from the viewpoint of delaying contact between the gas-liquid separation filter 150 and the liquid 110 as much as possible, the exhaust port 140 may be disposed at a position as far as possible from the introduction port 125 for the above-described liquid 110. For example, the exhaust port 140 may be disposed at a position where the straight line distance connecting the introduction port 125 and the exhaust port 140 is a maximum. For example, the gas-liquid separation filter 150 may be disposed to cover the exhaust port 140. In addition, the absorber 160 may be disposed to cover the gas-liquid separation filter 150.

Since the fluid device 100 shown in FIGS. 1B to 1D includes the absorber 160 or the like, the liquid 110 introduced into the tank 130 is absorbed by the absorber 160, and the liquid 110 does not block the exhaust port 140 immediately via the gas-liquid separation filter 150. As a result, irrespective of the direction in which the fluid device 100 is used (a direction of installation), the liquid 110 can be accommodated to the full capacity of the tank 130. Here, the direction (the installation direction) of the fluid device 100 is exemplified by, for example, a direction in which the exhaust port 140 is disposed upward (a direction opposite to that of gravity), a direction in which the exhaust port 140 is disposed downward (the direction of gravity), and a direction in which the exhaust port 140 is disposed laterally (a direction perpendicular to the direction of gravity).

Since the fluid device 100 of the embodiment includes both of the gas-liquid separation filter 150 and the absorber 160, a high filling rate of the liquid 110 into the tank 130 can be obtained by absorbing the liquid 110 using the absorber 160 in order to delay the time in which the liquid 110 contacts with the gas-liquid separation filter 150 or spatially delay a contact of the liquid 110 with the gas-liquid separation filter 150, or spatially reducing a contact area of the liquid 110 with the gas-liquid separation filter 150, while separating a gas and a liquid using the gas-liquid separation filter 150. Further, a gas flow path through which the gas in the second space 180 flows to the exhaust port 140 of the first space 170 can be easily secured.

In the fluid device of the embodiment, “the liquid 110 is accommodated fully to the capacity of the tank 130” includes a situation in which the liquid 110 is accommodated in the tank 130 as much as possible. An amount of the liquid 110 accommodated in the tank 130 may be, for example, 65% or more of the capacity of the tank, for example, 70% or more of the capacity of the tank, for example, 75% or more of the capacity of the tank, for example, 80% or more of the capacity of the tank, or for example, 85% or more of the capacity of the tank.

The material that constitutes the flow path or the tank of the fluid device is not particularly limited as long as the material is used for conventional fluid devices, and for example, polypropylene, polyethylene, polyisoprene, polybutadiene, polychloroprene, polyisobutylene, poly(styrene-butadiene-styrene), polyurethane, silicone polymers, poly(bis(fluoroalkoxy)phosphazene) (PNF, Eypel-F), poly(carborane-siloxane) (Dexsil), poly(acrylonitrile-butadiene) (nitrile rubber), poly(l-butene), poly(chlorotrifluoroethylene-vinylidene fluoride) copolymer (Kel-F), poly(ethylvinylether), poly(vinylidene fluoride), poly(vinylidene fluoride-hexafluoropropylene) copolymer (Viton), polyvinyl chloride (PVC) elastomer compositions, polysulfone, polycarbonate, polymethyl methacrylate (PMMA), polytetrafluoroethylene, chlorosilane, methylsilane, ethylsilane, phenylsilane, polydimethylsiloxane (PDMS), styrene-based polymers such as methacrylstyrene or the like, or the like, is exemplified.

### (Absorber)

In the fluid device of the embodiment, the absorber 160 can be used without particular limitation as long as the liquid (for example, a liquid having water as a main component, or the like) can be absorbed. The absorber is exemplified by, for example, a sponge, a woven fabric, a non-woven fabric, a foam structure, a porous polymer, a water absorptive polymeric compound, or the like. The absorber may be constituted by a plurality of layered structures formed of different materials (substances). For example, the absorber may be a structure body constituted by two polymer layers formed of different materials.

The absorber 160 is preferably processed using a surfactant or a wetting agent. When the absorber 160 is processed using a surfactant or/and the wetting agent, an absorption rate of the liquid 110 with respect to the absorber 160 tends to be able to be accelerated.

Here, processing using the surfactant or the wetting agent includes, for example, applying the surfactant or the wetting agent on the surface of the absorber 160, adding the surfactant or the wetting agent to the materials of the absorber 160, or the like. For example, after the absorber 160 is immersed in the surfactant or the wetting agent dissolved or dispersed in the solvent, the surfactant or the wetting agent can be applied to the surface of the absorber 160 by removing the solvent or the like.

The surfactant is exemplified by, for example, a fatty acid ester type non-ionic surfactant, a polyglycerin fatty acid ester, an alkyl ether sulfate, a higher alcohol sulfate, an alkyl phosphate metal salt, or the like. In addition, the wetting agent is exemplified by glycerin or the like. Regarding surfactants or wetting agents, one type thereof may be used alone, or two or more types thereof may be used in combination. In addition, the surfactant and the wetting agent may be mixed and used.

The absorber 160 preferably contains polyvinyl acetal, a polyvinyl alcohol or cellulose as a main component. Here, the polyvinyl acetal is exemplified by, for example, polyvinyl formal, polyvinyl butyral, or the like.

Here, containing as a main component means that 50 mass % or more, for example, 60 mass % or more, for example, 70 mass % or more, or for example 80 mass % or more of the absorber is composed of this component. The material may be used solely or two or more of the materials may be mixed and used. In the example, as described below, when the absorber 160 contains the above mentioned material as the main element, it tends to be easy to fully accommodate the liquid 110 to the capacity of the tank 130.

### (Gas-Liquid Separation Filter)

As described above, the fluid device of the embodiment includes a gas-liquid separation filter. A high filling rate of the fluid device regardless of the arrangement direction of the fluid device can be obtained since the gas liquid separation filter divides the inside of the tank into two spaces, the absorber is disposed in a space (a space on the side of the liquid) filled with the liquid and an exhaust port is disposed in a space (a space on the side of the gas) from which a gas is discharged.

Here, the gas-liquid separation filter includes a film body through which only a gas can pass and a liquid cannot pass or a liquid cannot easily pass. Since the fluid device of the embodiment includes the gas-liquid separation filter, a leakage of the liquid out of the fluid device can be minimized (or prevented). This is a performance required for a viewpoint of preventing an infectious disease when a living body specimen such as blood of a human is analyzed using the fluid device. The gas-liquid separation filter is preferably configured such that not only liquids but also infectious materials such as bacteria, viruses, or the like, cannot pass therethrough.

The material for the gas-liquid separation filter can be used without particular limitation as long as only a gas can pass therethrough and a liquid cannot pass therethrough or a liquid cannot easily pass therethrough, and may be, for example, a polytetrafluoroethylene film. The gas-liquid separation filter may be made of a material having water repellency, or it may be subjected to a water repellency treatment. For example, the filter may be processed to have a water repellent property by applying an organosilane on the surface of the filter, or the like.

The gas-liquid separation filter may be fixed to an inner wall of the tank 130 by, for example, an adhesive agent, or may be fixed to the inner wall through, for example, ultrasonic welding or the like. In addition, for example, a gas-permeable filter (for example, a gas-liquid separation filter) may also be disposed on the exhaust port to configure a double filter.

### (Fluid Device)

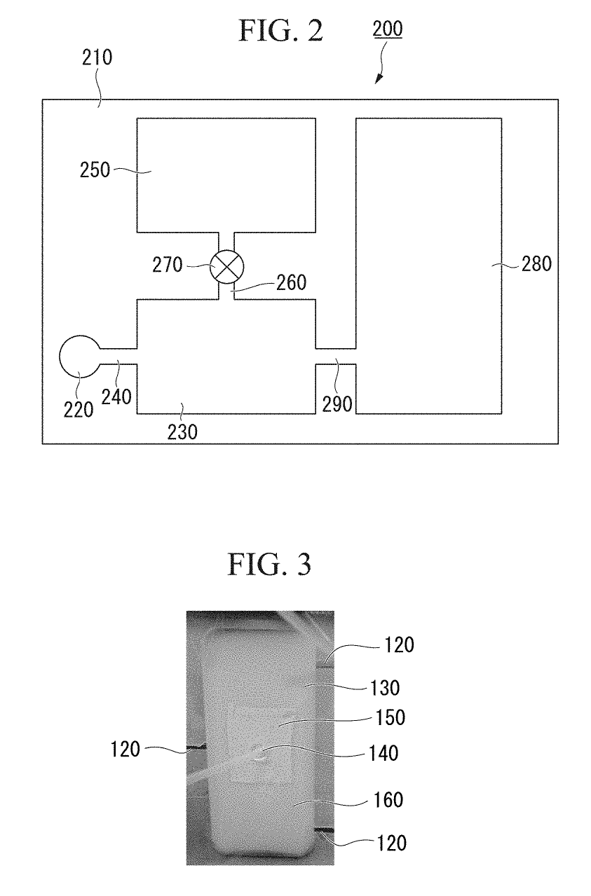

Hereinabove, while the tank portion of the fluid device has been described, here, the fluid device including the tank 130 will be described. FIG. 2 is a plan view schematically showing a fluid device according to the embodiment. A fluid device 200 is an example of the fluid device of the embodiment and configured to detect a target protein (a biomolecule, a particle, or the like, that is a detection target) in a specimen. The fluid device of the embodiment is not limited to the fluid device 200.

The fluid device 200 shown in FIG. 2 includes an inlet 220, a detector 230, a flow path 240 that connects the inlet 220 and the detector 230, a reservoir 250, a flow path 260 that connects the reservoir 250 and the detector 230, a valve 270 configured to control a flow of a fluid in the flow path 260, a tank 280, and a flow path 290 that connects the detector 230 and the tank 280, which are formed on a base plate 210. The tank 280 has the same configuration as the above-mentioned tank 130. For example, the materials of the fluid device 200 are exemplified by the same materials as for the above-mentioned flow path or the above-mentioned tank 130.

Next, a process of detecting a target protein using the fluid device 200 will be described. In an initial state, a first specific bonding material specific to the target protein is fixed to a predetermined surface of the detector 230, a reagent containing a second specific bonding material specific to the target protein is accommodated in the reservoir 250, and the valve 270 is closed. The second specific bonding material accommodated in the reservoir 250 is marked with, for example, a fluorochrome.

First, a sample (a specimen) such as blood serum, blood plasma, or the like, is introduced into the inlet 220. The sample introduced into the inlet 220 is introduced into the detector 230 through the flow path 240. The target protein in the sample is captured by the first specific bonding material disposed on the inner wall (the predetermined surface) of the detector 230. Here, an antigen, an antibody, an aptamer, or the like, is exemplified as the specific bonding material. The sample that has passed through the detector 230 is accommodated in the tank 280 as a waste liquid via the flow path 290.

Then, a cleansing liquid is introduced from the inlet 220. The cleansing liquid introduced into the inlet 220 is introduced into the detector 230 through the flow path 240. Impurities present in the detector 230 are removed by the cleansing liquid and accommodated in the tank 280 through the flow path 290 as a waste liquid.

Next, the valve 270 is released. Accordingly, a reagent containing the second specific bonding material accommodated in the reservoir 250 is introduced into the detector 230 through the flow path 260. As a result, the second specific bonding material marked with fluorochrome is bonded to the target protein captured by the first specific bonding material on the inner wall of the detector 230. The second specific bonding material, which has not reacted, passing through the detector 230 is accommodated in the tank 280 through the flow path 290.

Then, the cleansing liquid is introduced from the inlet 220. The cleansing liquid introduced into the inlet 220 is introduced into the detector 230 through the flow path 240. Impurities present in the detector 230 are removed by a cleansing liquid and accommodated in the tank 280 through the flow path 290 as a waste liquid.

Next, excitation light is radiated to the detector 230 from the detection device and an intensity of generated fluorescence is measured by the detection device. Accordingly, a target protein in the sample can be detected (for example, quantified). Measurement of the intensity of the fluorescence is performed by, for example, a controller of such as a fluorescence microscope, a light source, a personal computer, and so on (not shown).

According to the fluid device of the embodiment, the liquid such as a waste liquid or the like can be fully accommodated to the capacity of the tank 280. Further, the fluid device of the embodiment may have, for example, a configuration in which a flow path (for example, a mixing section including a loop-shaped flow path configured to mix a plurality of solutions, or the like) configured to mix or quantify a plurality of solutions (for example, a specimen including a reagent and a target protein) is provided for preprocessing, and the flow path and the detector 230 are connected to each other.

### EXAMPLES

Next, while the embodiment will be described using examples, the present invention is not limited to the following examples.

Fluid devices of Comparative Example and Experimental Examples 1 to 4 were manufactured. The fluid device of Comparative example includes a tank, and the tank includes an exhaust port, and a gas-liquid separation filter disposed in the tank to cover the exhaust port. The fluid device of each of Experiment examples 1 to 4 includes a tank (for example, the tank 130), and the tank includes an exhaust port, a gas-liquid separation filter disposed in the tank to cover the exhaust port, and an absorber (for example, the absorber 160) disposed in the tank.

In the fluid device of each of Comparative example Experiment examples 1 to 4, a polytetrafluoroethylene film (a hole diameter of 100 μm, a thickness of 100 μm, a water pressure resistance of 60 kPa) was used as the gas-liquid separation filter. The gas-liquid separation filter was disposed at a position closer to the exhaust port than a position at which the absorber is disposed.

The fluid device of Comparative example had no absorber, and the inside of the tank was a void.

In addition, in the fluid device of Experiment example 1, sponge (an average hole diameter of the sponge is 130 μm) formed of polyvinyl formal was used as the absorber. In addition, a water retention represented by an own weight ratio when water is maximally absorbed in the sponge was 10.9.

In addition, in the fluid device of Experiment example 2, sponge (an average hole diameter of the sponge is 80 μm) formed of polyvinyl formal was used as the absorber. In addition, a water retention represented by an own weight ratio when water is maximally absorbed in the sponge was 9.7.

In addition, in the fluid device of Experiment example 3, sponge (an average hole diameter of the sponge is 60 μm) having a core formed of polyester and a surface formed of polyvinyl alcohol was used as the absorber. In addition, a water retention represented by an own weight ratio when water is maximally absorbed in the sponge was 6.1.

In addition, in the fluid device of Experiment example 4, non-woven fabric formed of a material having cellulose and cotton was used as an absorber. In addition, a water retention represented by an own weight ratio when water is maximally absorbed in the non-woven fabric was 12.3.

Next, a liquid (colored water) was introduced into the tank of the fluid device of each of Comparative example and Experiment examples 1 to 4, and an amount of the liquid that can be accommodated in the tank was reviewed. The fluid device was used in a direction in which the exhaust port is disposed laterally (a direction perpendicular to the direction of gravity). In addition, introduction of the liquid into the tank was performed by connecting the exhaust port to the suction pump and performing suctioning.

Table 1 represents properties and test results of the used absorber. In Table 1, “PVFM” represents polyvinyl formal, and “PVA” represents polyvinyl alcohol. A water retention in the absorber represents an own weight ratio when the water is maximally absorbed by the absorber. An amount of the liquid accommodated in the tank represents a filling rate (%) of the tank.

As shown in Table 1, a tank filling rate in the fluid device of Comparative example was 63.7%. On the other hand, a tank filling rate in the fluid device of Experiment example 1 was 85.3%, which was about 1.34 times a tank filling rate of the fluid device of Comparative example.

In addition, a tank filling rate in the fluid device of Experiment example 2 was 88.0%, which was about 1.38 times a tank filling rate of the fluid device of Comparative example.

In addition, a tank filling rate in the fluid device of Experiment example 3 was 82.1%, which was about 1.28 times a tank filling rate of the fluid device of Comparative example.

In addition, a tank filling rate in the fluid device of Experiment example 4 was 81.7%, which was about 1.28 times a tank filling rate of the fluid device of Comparative example.

In this way, in the fluid device of each of Experiment examples 1 to 4 in which the absorber is disposed in the tank, even when the fluid device is used in a direction in which the exhaust port is disposed laterally (a direction perpendicular to the direction of gravity), it was found that the liquid can be accommodated at a higher tank filling rate than in the fluid device of Comparative example.

FIG. 3 is a representative photograph showing a tank portion of the fluid device of Experiment example. As shown in FIG. 3, the fluid device of Experiment example includes the tank 130, and the tank 130 includes the exhaust port 140, the gas-liquid separation filter 150 disposed in the tank to cover the exhaust port, and the absorber 160 disposed in the tank.

TABLE 1

| | Absorbent | | | |
|---|---|---|---|---|
| | | Hole | Water | Tank |
| | | diameter | retention (own | filling |
| | Material | (μm) | weight ratio) | rate (%) |
| Comparative | — | — | — | 63.7 |
| Example | | | | |
| Experiment | PVFM | 130 | 10.9 | 85.3 |
| example 1 | | | | |
| Experiment | PVFM | 80 | 9.7 | 88.0 |
| example 2 | | | | |
| Experiment | Core material: | 60 | 6.1 | 82.1 |
| example 3 | polyester | | | |
| | Surface: PVA | | | |
| Experiment | Cellulose, cotton | — | 12.3 | 81.7 |
| example 4 | | | | |

What is claimed is:

1. A fluid device including a flow path through which a liquid flows, the fluid device comprising:
a tank connected to the flow path and in which the liquid is accommodated,
wherein the tank comprises:
an exhaust port from which a gas is discharged;
a gas-liquid separation filter that suppresses outflow of the liquid from the exhaust port; and
an absorber that is disposed in the tank and that absorbs the liquid.

2. The fluid device according to claim 1, wherein the gas-liquid separation filter is disposed at a position of the exhaust port or in a vicinity of the exhaust port.

3. The fluid device according to claim 1, wherein at least a portion of the absorber is disposed at a position that overlaps the gas-liquid separation filter when seen in a direction perpendicular to a surface on which the exhaust port is formed.

4. The fluid device according to claim 1, wherein the inside of the tank is divided into a first space and a second space by the gas-liquid separation filter, and
the exhaust port is installed in the first space and the absorber is disposed in the second space.

5. The fluid device according to claim 1, wherein the gas-liquid separation filter is disposed at a position closer to the exhaust port than a position at which the absorber is disposed.

6. The fluid device according to claim 1, wherein the absorber and the gas-liquid separation filter have a positional relation in which, among the absorber and the gas-liquid separation filter, the absorber comes in contact with the liquid earlier than the gas-liquid separation filter.

7. The fluid device according to claim 1, wherein the absorber contains water absorptive polymer.

8. The fluid device according to claim 1, wherein the absorber is disposed to face a surface of the gas-liquid separation filter through which the gas passes.

9. The fluid device according to claim 1, wherein the absorber is disposed to cover one surface of a film body in the gas-liquid separation filter.

10. The fluid device according to claim 1, comprising a suction port connected to a suction section that suctions a gas in the tank from an outside of the tank,
wherein the exhaust port is connected to the suction port.

11. The fluid device according to claim 1, wherein the tank comprises an introduction port through which a liquid flowing through the flow path flows into the tank.

12. The fluid device according to claim 11, wherein, in a direction in which the liquid flows from the introduction port toward the exhaust port, the absorber and the gas-liquid separation filter are disposed in sequence of the absorber and the gas-liquid separation filter from a side close to the introduction port.

13. The fluid device according to claim 11, wherein, in a direction perpendicular to a surface on which the exhaust port is formed, at least a part of the absorber is disposed in a vicinity of the introduction port.

14. The fluid device according to claim 11, wherein the absorber is disposed in a vicinity of the introduction port.

15. The fluid device according to claim 1, wherein the absorber is processed with a surfactant or a wetting agent.

16. The fluid device according to claim 1, wherein the absorber contains polyvinyl acetal, polyvinyl alcohol or cellulose as a main component.

17. The fluid device according to claim 1, wherein the gas-liquid separation filter is a polytetrafluoroethylene film.

18. A fluid device including a flow path, the fluid device comprising:
a tank that includes an exhaust port from which a gas is exhausted and an introduction port connected to the flow path, and in which a liquid flowing from the introduction port is accommodated;
a gas-liquid separation filter that is disposed at a position of the exhaust port or in a vicinity of the exhaust port and that suppresses outflow of the liquid; and
an absorber that absorbs the liquid,
wherein the tank is divided into a first side into which the liquid flows and a second side from which the gas is exhausted, with reference to the gas-liquid separation filter, and
the absorber is disposed at the first side of the tank.

19. The fluid device according to claim 18, wherein the exhaust port is disposed on the second side.
